(19) United States
(12) Patent Application Publication
(10) Pub. No.: US 2022/0095749 A1
(43) Pub. Date: Mar. 31, 2022

(54) Strap for a wearable electronic device and a wearable electronic device comprising the same

(71) Applicant: Suunto Oy, Vantaa (FI)

(72) Inventors: Britta Stockinger, Vantaa (FI); Jere Lallo, Vantaa (FI); Jouni Junes, Vantaa (FI); Atte Kuparinen, Vantaa (FI); Kimmo Pernu, Vantaa (FI)

(21) Appl. No.: 17/369,250

(22) Filed: Jul. 7, 2021

(30) Foreign Application Priority Data
Sep. 30, 2020 (FI) 20205952

(51) Int. Cl. A44C 5/00; A44C 5/16; A44C 5/20; D04H 1/4358

(52) U.S. Cl. CPC A44C 5/0069; A44C 5/16; D10B 2401/061; D04H 1/4358; A44C 5/2033

(57) ABSTRACT

According to an example aspect of the present invention, there is provided a strap for a wearable electronic device. The strap features two mutually superposed layers of a strip having microfiber base material.

### FIELD

The present disclosure relates to apparatuses affixing a wearable electronic device to the user's anatomy. In particular the disclosure relates to straps for the purpose of providing a snug fit for an electronic wearable device comprising an optical heart rate sensor in order to improve heart rate measurements.

### BACKGROUND

The fit of a strap for a wearable electronic device is important not only for providing a comfortable using experience but also for ensuring reliable operation of the device. Several attempts have been made to improve the fit of a strap by optimizing its stretching properties, as discussed in US 20160255921 A1 and US20170065038A1.

There remains, however, a need to further explore the possibilities of perfecting the fit of a strap for a wearable electronic device or at least to provide the public with a useful alternative.

### SUMMARY

The invention is defined by the features of the independent claims. Some specific embodiments are defined in the dependent claims.

According to a first aspect of the present disclosure, there is provided a strap for a wearable electronic device. The strap features two mutually superposed layers of a strip having microfiber base material.

According to a second aspect of the present disclosure, there is provided a wearable electronic device with such a strap.

Certain embodiments of the invention may include one or more features from the following list:
the microfiber base material is an ultra-microfiber fabric;
the microfiber base material is Ultrasuede®;
the blank of the strip consists of a single piece ultra-microfiber fabric folded as two mutually superposed layers;
the strip comprises an inner layer;
the strip comprises an outer layer;
the outer layer is superposed and attached onto the inner layer;
the strip comprises a loop section;
the loop section connects the inner layer to the outer layer and forms a space for a fixture to the wearable electronic device;
the strip is elongated along a longitudinal dimension;
the strip has a width in a transversal dimension in respect to the longitudinal dimension:
the strip exhibits greater elasticity in the longitudinal dimension than in the transversal dimension;
the difference in elasticity between the longitudinal dimension and the transversal dimension is 20% or more;
the difference in elasticity between the longitudinal dimension and the transversal dimension is 50% or more;
the difference in elasticity between the longitudinal dimension and the transversal dimension is 100% or more;
the difference in elasticity between the longitudinal dimension and the transversal dimension is 200% or more;
the difference in elasticity between the longitudinal dimension and the transversal dimension is by one order of magnitude or more;
the strap comprises a first part having an attachment section and a buckle section;
the strap comprises a second part having an attachment section and a tip section;
the attachment section of the first part is configured to attach to the wearable electronic device at a first attachment point on the wearable electronic device;
the buckle section of the first part is configured to receive the tip section of the second part there through;
the buckle section of the first part is configured to attach to the second part;
the attachment section of the second part is configured to attach to the wearable electronic device at a second attachment point on the wearable electronic device;
the strap comprises a retractable spring bar as the fixture inserted into the loop section of the strip;
the inner layer and the outer layer have been glued to each other;
the strap comprises stitching in the vicinity of the buckle section of the first part or the loop section of the first part or second part or in any combination thereof;
the first part comprises a buckle and a cooperative tang at the buckle section;
the second part comprises a corresponding tip which is configured to be inserted through the buckle;
the second part comprises a plurality of holes for receiving the tang;
the first part comprises a loop;
the second part comprises a tip which is configured to be inserted through the loop;
the strap comprises a Velcro interface between the first part and the second part for affixing the first part to the second part;
the wearable electronic device comprises an enclosure;
the enclosure comprises a first attachment point;
the first attachment point is provided at one end of the enclosure;
the first attachment point is configured to receive the loop section of the first part of the strap through a fixture between the enclosure and the loop section;
the enclosure comprises a second attachment point;
the second attachment point is provided at another end of the enclosure in respect to the first attachment point;
the attachment point is configured to receive the loop section of the second part of the strap through a fixture between the enclosure and the loop section;
the attachment points are both configured to receive a spring bar,
the wearable electronic device comprises an optic heart rate sensor housed in the enclosure.

Considerable benefits are gained with aid of the present invention. By using microfiber material as the raw material of the strap, elasticity is gained in one dimension and lost in another. This enables orientation of the strap fabric such that, once closed into a loop, the elasticity of the strap ensures a reliable fit of the wearable electronic device on the user. The relatively soft microfiber material of the strap, in turn, facilitates compliance with minor contours on the users anatomy, such as protruding bones, etc. By having relatively little elasticity in the transversal dimension, the strap maintains a good deformation resistance. Such benefits are particularly useful in applications in which the wearable electronic device features an optical heart rate sensor, the accuracy of which is greatly dependent on a uniform contact with the user's skin. Optical heart rate or pulse measurement is performed with a sensor arrangement with light emitters and light sensors placed at the watch case bottom. The measurement is disturbed by ambient light reaching the sensor or sensors from the sides if the watch does not stay in good contact with the skin. Also the oscillations or vibrations of the wrist tissue at the sensors from when the wrist is moving, for example when running, causes signal noise thus making the measurement more difficult. The problems with the reliability of the measurement are worsened if the watch is relatively heavy and loosely fit. On the other hand, an overly tight watch strap would be too uncomfortable. It is therefore desirable to have a snug fit with a comfortable feel in order to make a desirable product with good quality. In other words, the novel strap construction has the potential in improving the accuracy of heart rate signal acquired with optical heart rate sensors enclosed in a wearable electronic device.

### BRIEF DESCRIPTION OF THE DRAWINGS

In the following certain exemplary embodiments are described in greater detail with reference to the accompanying drawings, in which:

FIG. 1 illustrates a top elevation view of a strap in accordance with at least some embodiments of the present invention connected to an electronic wearable device shown in dashed lines in a flat configuration;

FIG. 2A illustrates a top elevation view of the short piece of the strap of FIG. 1;

FIG. 2B illustrates a side elevation view of the short piece of the strap of FIG. 1;

FIG. 2C illustrates a bottom elevation view of the short piece of the strap of FIG. 1,

FIG. 3A illustrates a top elevation view of the long piece of the strap of FIG. 1;

FIG. 3B illustrates a side elevation view of the long piece of the strap of FIG. 1;

FIG. 3C illustrates a bottom elevation view of the long piece of the strap of FIG. 1, and

FIG. 4 illustrates a side elevation view of the strap and electronic wearable device of FIG. 1 in a loop configuration.

### EMBODIMENTS

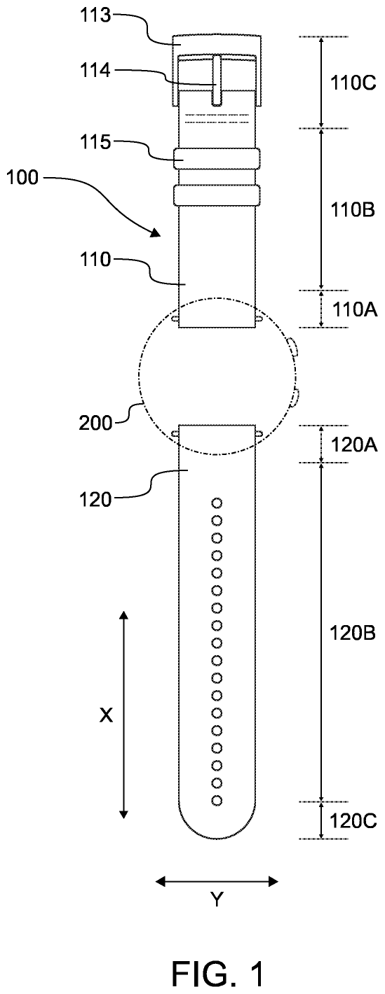

FIG. 1 illustrates an exemplary strap 100 attached to a wearable electronic device 200 shown in dashed line according to an embodiment. The strap and wearable electronic device 200 is presented in a flat configuration with the strap extending straight from the wearable electronic device. The embodiment is described in the form of a wrist band for affixing a smart watch to the wrist of a user. The principles herein described and defined in the appended claims are applicable to other terminal devices, such as heart rate monitors or motion sensors, affixed to different parts of human anatomy, such as the chest, ankle, or upper arm. Furthermore the illustrated examples present a buckle mechanism for attaching the strap pieces 110, 120 to each other but the buckle mechanism could be replaced with a different closing mechanism, such as a Velcro attachment, a butterfly clasp, etc. The embodiments share the central purposeful concept of the strap 100 or strap pieces 110, 120 being made of a blank strip of microfiber, particularly ultra-microfiber, such as Ultrasuede®.

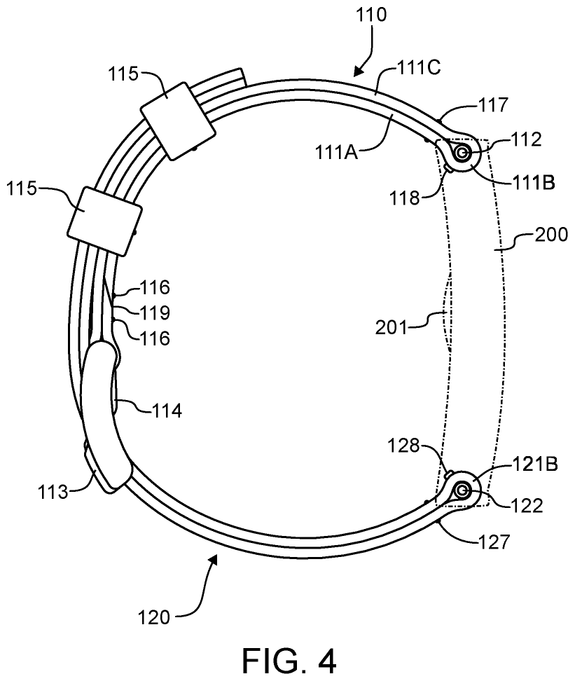

Let us first turn to the strap 100 which is shown in a planar spread-out configuration in FIG. 1 and in a loop configuration in FIG. 4. The strap 100 is made up by two parts, namely a first part 110 and a second part 120. The wearable electronic device 200, such as a smart watch, is provided and affixed to between the parts 110, 120. The first part 110 acts as the female part of a two-part strap and comprises a buckle 113 with a tang 114 for engaging a respective opening in the second part 120. In the present field, such strap parts are referred to as “short pieces”. As indicated above, the buckle 113 could be replaced with a different mechanism, such as a simple loop for a Velcro attachment (not illustrated in the FIGURES), a clasp part (not illustrated in the FIGURES) or a positively engaging quick coupler (not illustrated in the FIGURES). The first part 110 has three sections, namely an attachment section 110A at one end for attachment to the wearable electronic device 200, a buckle section 110C at the other end for accommodating the attachment to the second strap part 120, and an intermediate section 110B there between. The first part 110 may also include conventional loops 115 for keeping excess sections of the second strap part 120 in a loop configuration of the strap (FIG. 4).

The second part 120 acts as the male part of a two-part strap and comprises a a tip 123 for entering through the buckle 113 and a sequence of holes 124 for receiving the a tang 114. In the present field, such strap parts are referred to as “long pieces”. As indicated above, the buckle mechanism could be replaced with an alternative, whereby the second part 120 would include a Velcro attachment piece, a magnet, a clasp part, a quick coupler, etc. The second part 120 has three sections, namely an attachment section 120A at one end for attachment to the wearable electronic device 200, a tip section 120C at the other end for accommodating the attachment to the first strap part 110, and an intermediate section 120B there between.

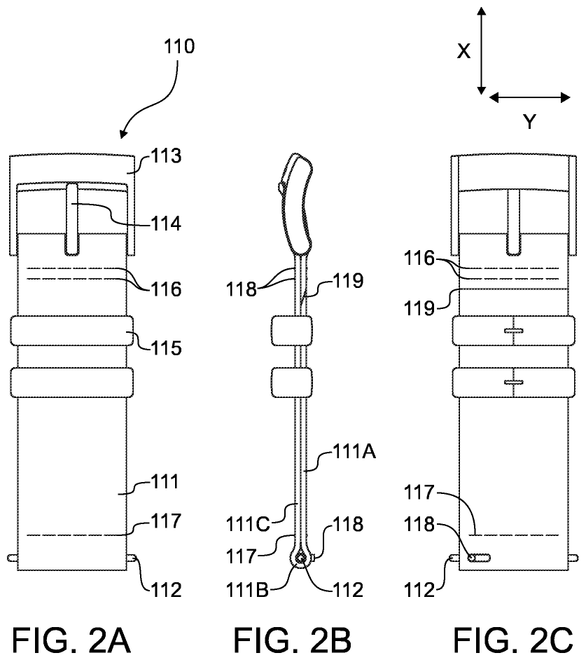

FIGS. 2A, 2B, and 2C show details of the first part 110. FIG. 2B shows the first part 110A from the side revealing the construction of the strap. The first strap part 110 is made from a strip 111 of microfiber. To be more precise from a blank of microfiber may be laminated into two layers with elastic glue or heat activated bonding layer and then cut to the final shape or the strip is first cut from a blank of microfiber which is then processed into the shape shown in the FIGURES. In other words, the strip 111 comprises microfiber base material. The strip 111, when processed into a strap 110, may include further materials, such as glue or stitching yarn, but the base of the strip is formed by a microfiber cloth. The strip 111 is elongated along a longitudinal dimension X, has a width in a first transversal dimension Y and a thickness in another transversal dimension. The microfiber material is preferably ultra-microfiber, such as Ultrasuede®. The microfiber material comprises polyester and polyurethane. According to a particular embodiment the microfiber comprises 65 to 80 weight-% of polyester ultra-microfiber which is non-woven with 35 to 20 weight-% of non-fibrous polyurethane binder.

The strip 111 extends along the longitudinal dimension X to form an inner layer 111A. The strip 111 is oriented such that it exhibits greater elasticity in the longitudinal dimension X than in the transversal dimension Y. The difference in the elastic modulus between the longitudinal dimension X and the transversal dimension Y may be 20% or more, preferably 50% or more, 100% or more, 200% or more, or by more than one or two order of magnitude. The strip 111 features a parallel outer layer 111C similarly oriented so as to maintain the orientation of the stretching ability along the longitudinal dimension X. The strip 111 transitions between the inner layer 111A and outer layer 111C at a loop section 111B at the attachment section of the first part 110A and at another loop (covered by the buckle in FIG. 2B) at the buckle section 110C. In other words the strip 111 is wound into two superposed layers 111A, 111C. The layers 111A, 111C are attached to each other by gluing for example with a thermosetting adhesive there between. The attachment may be secure at the vicinity of the loop sections 111B with stitching 117.

Any layer of material, such as adhesive, between the inner layer and outer layer is thinner than the inner layer or the outer layer. In particular, the intermediate layer between the inner layer and outer layer has a thickness of preferably 50% or less, more preferably 25% or less, of the thickness of the inner layer or the outer layer.

The first part houses a spring bar 112 within the strip 111 enclosed by the loop section 111B. The spring bar 112 is used for attachment to the wearable electronic device 200. The spring bar 112 may be operated with a release mechanism 118 for toggling the movable pin of the spring bar 112 between a deployed and retracted state. The release mechanism 118 may be accessible through a respective opening provided to the strip 111. The buckle 113 comprises a comparable bar (not shown in the FIGURES), around which the loop section of the strip 111 is wound. The preferably beveled ends of the strip 111 meet at a seam 119 which is closed by gluing. According to another embodiment, the ends of the strip 111 at the seam 119 is closed by a thermoset adhesive. According to another embodiment, the ends of the strip 111 at the seam 119 is closed by contact glue. According to another embodiment, the ends of the strip 111 at the seam 119 is closed by welding. According to another embodiment, the ends of the strip 111 at the seam 119 is closed by sewing. According to another embodiment, the ends of the strip 111 at the seam 119 is closed by melting the layers together by applying heat.

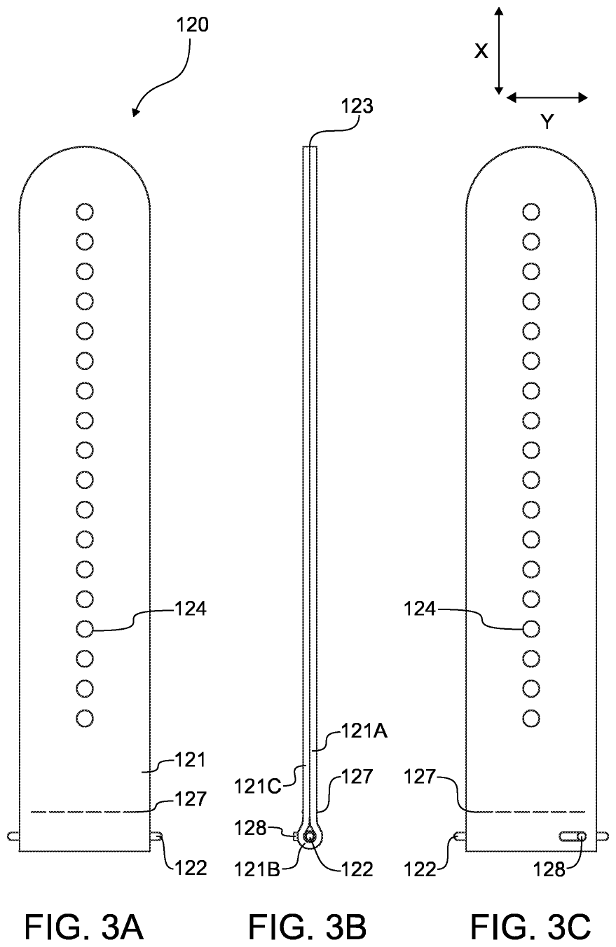

FIGS. 3A, 3B, and 3C show details of the second part 120. FIG. 2B shows the first part 110A from the side revealing the construction of the strap which is largely similar to that of the first part 110. The second strap part 120 is made from an elongated strip 121 of microfiber. The strip 121 extends along the longitudinal dimension X to form an inner layer 121A such oriented that it exhibits greater elasticity in the longitudinal dimension X than in the transversal dimension Y. The difference in the elastic modulus between the longitudinal dimension X and the transversal dimension Y may be 20 or more, 50% or more, 100% or more, 200% or more or by one or two order of magnitude or more. The strip 121 features a parallel outer layer 121C similarly oriented so as to maintain the orientation of the stretching ability along the longitudinal dimension X. The strip 121 transitions between the inner layer 121A and outer layer 121C at a loop section 121B at the attachment section 120A of the second part 120 for housing a spring bar 122. The ends of the strip 121 meet at the tip 123 at the tip section 120C. In other words the strip 121 is wound into two superposed layers 121A, 121C. The layers 121A, 112C are attached to each other by gluing for example with a thermosetting adhesive there between. The attachment may be additionally secured at the vicinity of the loop section 121B and/or at the tip 123 with stitching 127, thermoset gluing, welding, or sewing. The tip section 120C may also include stitching for preventing the ends of the strip 121 from becoming detached from each other. A series of subsequently positioned holes 124 have been provided through the strip 121 along the longitudinal dimension for receiving the tang 114 of the buckle 113 of the first part 110A.

The exemplary wearable electronic device 200 takes the form a smart watch. The enclosure of the wearable electronic device 200 includes two attachment points at opposing ends of the enclosure; one for each spring bar 112, 122 of the strap parts 110, 120. Naturally, the spring bar attachment could be replaced with other foreseeable attachment mechanisms, such as affixer-secured or clenched bars, sliding coupler parts in a corresponding attachment groove on the enclosure, magnets, etc.

The enclosure of the wearable electronic device 200 preferably also includes an optical heart rate sensor 201, whereby the benefits of the novel strap may be utilized for the purpose of ensuring a reliable fit between the wrist of the user and the sensor optics. Optical heart rate or pulse measurement is performed with a sensor arrangement with light emitters and light sensors placed at the watch case bottom. The measurement is disturbed by ambient light reaching the sensor or sensors from the sides if the watch does not stay in good contact with the skin. Also the oscillations or vibrations of the wrist tissue at the sensors from when the wrist is moving, for example when running, causes signal noise thus making the measurement more difficult. The problems with the reliability of the measurement are worsened if the watch is relatively heavy and loosely fit. On the other hand, an overly tight watch strap would be too uncomfortable. It is therefore desirable to have a snug fit with a comfortable feel in order to make a desirable product with good quality.

The use of the strap 100 is straight-forward. The wearable electronic device 200 is placed on the desired anatomic location of the user, such as the wrist. The strap parts 110, 120 are coupled to each other by inserting the tip 123 through the buckle 114, by pulling a desired amount of tension into the strap 100 and securing the strap into a loop around the anatomic location by inserting the tang 114 into a corresponding hole 124 of the second strap part 120. Once closed into a loop, the elasticity of the strap 100 along the longitudinal dimension X ensures a reliable fit of the wearable electronic device 200 on the user. The microfiber material of the strap 100, in turn, facilitates compliance with minor contours on the users anatomy, such as protruding bones, etc. By having relatively little elasticity in the transversal dimension, the strap maintains a good deformation resistance. The relatively small stretch in the transversal dimension Y, i.e. along the width of the strap, facilitates sturdy attachment to the hardware of the device, e.g. to the spring bar and buckle. If the strap would be relatively compliant in the transversal dimension Y, the excess elasticity could compromise attachment to the wearable electronic device. The relative resistance to elastic deformation in the transversal dimension Y minimizes fatigue in the adhesive layer between the strap layers 111A, 111C; 121A, 121C.

The strap 100 may be further enhanced by including a reflective yarn pattern, an embedded auxiliary battery, etc. The base material of the strap may be treated with a anti-bacterial supplement for making the strap more suitable for a sporting device application.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

REFERENCE SIGNS LIST

| No. | Feature |
|---|---|
| 100 | strap |
| 110 | first part, “short piece” |
| 110A | attachment section |
| 110B | intermediate section |
| 110C | buckle section |
| 111 | strip |
| 111A | inner layer |
| 111B | loop section |
| 111C | outer layer |
| 112 | spring bar |
| 113 | buckle |
| 114 | tang |
| 115 | loop |
| 116 | stitching for buckle |
| 117 | stitching for spring bar |
| 118 | release mechanism |
| 119 | seam |
| 120 | second part, “long piece” |
| 120A | attachment section |
| 120B | intermediate section |
| 120C | tip section |
| 121 | strip |
| 121A | inner layer |
| 121B | loop section |
| 121C | outer layer |
| 122 | spring bar |
| 123 | tip |
| 124 | hole |
| 127 | stitching for spring bar |
| 128 | release mechanism |
| 200 | wearable electronic device |
| 201 | optical heart rate sensor |
| X | longitudinal dimension |
| Y | transversal dimension |

### CITATION LIST

### Patent Literature

US 20160255921 A1
US 20170065038 A1

1. A strap for a wearable electronic device, the strap comprising two mutually superposed layers of a strip, wherein:
the strip is elongated along a longitudinal dimension and has a width in a transversal dimension,
the strip comprises microfiber base material, and wherein
the strip exhibits greater elasticity in the longitudinal dimension than in the transversal dimension.

2. The strap according to claim 1, wherein the microfiber base material is an ultra-microfiber fabric, such as Ultrasuede®.

3. The strap according to claim 1, wherein the blank of the strip consists of a single piece ultra-microfiber fabric folded as two mutually superposed layers.

4. The strap according to claim 1, wherein the strip comprises:
an inner layer;
an outer layer superposed and attached onto the inner layer, and
a loop section connecting the inner layer to the outer layer and forming a space for a fixture to the wearable electronic device.

5. The strap according to claim 1, wherein the difference in elasticity between the longitudinal dimension and the transversal dimension is:
20% or more.

6. The strap according to claim 1, wherein:
the strap comprises:
a first part having an attachment section and a buckle section, and
a second part having an attachment section and a tip section,
the attachment section of the first part (his configured to attach to the wearable electronic device at a first attachment point on the wearable electronic device,
the buckle section of the first part is configured to:
receive the tip section of the second part there through, and to
attach to the second part, and wherein
the attachment section of the second part is configured to attach to the wearable electronic device at a second attachment point on the wearable electronic device.

7. The strap according to claim 4, wherein the strap further comprises a retractable spring bar as the fixture inserted into the loop section of the strip.

8. The strap according to claim 1, wherein the inner layer and the outer layer have been glued to each other.

9. The strap according to claim 6, wherein the strap further comprises stitching in the vicinity of the buckle section of the first part or the loop section of the first part or second part or in any combination thereof.

10. The strap according to claim 6, wherein:
the first part comprises a buckle and a cooperative tang at the buckle section, and wherein
the second part comprises a corresponding tip which is configured to be inserted through the buckle and comprises a plurality of holes for receiving the tang.

11. The strap according to claim 6, wherein:
the first part comprises a loop;
the second part comprises a corresponding tip, which is configured to be inserted through the loop, and wherein
the strap comprises a Velcro interface between the first part and the second part for affixing the first part to the second part.

12. A wearable electronic device comprising a strap, wherein the strap comprises two mutually superposed layers of a strip, wherein:
the strip is elongated along a longitudinal dimension and has a width in a transversal dimension,
the strip comprises microfiber base material, and wherein
the strip exhibits greater elasticity in the longitudinal dimension than in the transversal dimension.

13. The wearable electronic device according to claim 12, wherein the wearable electronic device further comprises an enclosure with:
a first attachment point provided at one end of the enclosure, which first attachment point is configured to receive the loop section of the first part of the strap through a fixture, and
a second attachment point provided at another end of the enclosure, which second attachment point is configured to receive the loop section of the second part of the strap through a fixture.

14. The wearable electronic device according to claim 13, wherein the attachment points are both configured to receive a spring bar.

15. The wearable electronic device according to claim 13, wherein the wearable electronic device further comprises an optic heart rate sensor housed in the enclosure.

16. The strap according to claim 1, wherein the difference in elasticity between the longitudinal dimension and the transversal dimension is 50% or more.

17. The strap according to claim 1, wherein the difference in elasticity between the longitudinal dimension and the transversal dimension is 100% or more.

18. The strap according to claim 1, wherein the difference in elasticity between the longitudinal dimension and the transversal dimension is 200% or more.
